(19) United States
(12) Patent Application Publication
(10) Pub. No.: US 2017/0173180 A1
(43) Pub. Date: Jun. 22, 2017

(54) CANCER IMMUNOTHERAPY COMPOSITIONS AND METHODS

(71) Applicant: THE REGENTS OF THE UNIVERSITY OF MICHIGAN, Ann Arbor, MI (US)

(72) Inventor: Qiao LI, Canon, MI (US)

(73) Assignee: THE REGENTS OF THE UNIVERSITY OF MICHIGAN, Ann Arbor, MI (US)

(21) Appl. No.: 15/129,194

(22) PCT Filed: Mar. 27, 2015
(86) PCT No.: PCT/US2015/022909

§ 371 Date: Sep. 26, 2016

Related U.S. Application Data
(60) Provisional application No. 61/971,318, filed on Mar. 27, 2014.

(51) Int. Cl. A61K 48/00; C12N 5/0781; C12Q 1/70; A61K 35/17

(52) U.S. Cl. CPC A61K 48/005; A61K 35/17; C12N 5/0635; C12Q 1/70; C12N 2510/00; C12N 2501/231; A61K 2035/124

(57) ABSTRACT

The present invention relates to compositions and methods for cancer immunotherapy. In particular, the present invention relates to engineered effector B cells and their use in cancer immunotherapy.

### STATEMENT REGARDING FEDERALLY SPONSORED RESEARCH OR DEVELOPMENT

This invention was made with government support under CA082529 awarded by the National Institutes of Health. The government has certain rights in the invention.

### FIELD OF THE INVENTION

The present invention relates to compositions and methods for cancer immunotherapy. In particular, the present invention relates to engineered effector B cells and their use in cancer immunotherapy.

### BACKGROUND OF THE INVENTION

Clinical trials to treat patients with cancer using adoptively transferred T cells or dendritic cells (DC) have shown therapeutic efficacy for patients with advanced diseases. However, the clinical responses to such immunotherapeutic approaches have been confined to a limited percentage of treated patients. Generally, bulk tumor masses with heterogeneous populations of cancer cells have been used as a source of antigen either to generate effector T cells or to prime DC vaccines. Human tumors are composed of heterogeneous tumor cell clones that differ with respect to proliferation, differentiation, and ability to initiate daughter tumors. Immunotherapy has become a viable treatment alternative for a number of advanced hematological malignancies and solid tumors (Mellman, I., et al., 2011. Nature 480: 480-489). Historically, studies of adoptive immunotherapy have focused on the generation of effector T cells against tumors. Adoptive transfer of tumor-reactive T cells has become a promising strategy for cancer immunotherapy (Ward, B. A., et al., 1988. J Immunol 141: 1047-1053; Chang, A. E., et al., 1989. Cell Immunol 120: 419-429; Geiger, J. D., et al., 1993. J Immunother Emphasis Tumor Immunol 13: 153-165; Li, Q., et al., 2005. J Immunol 175: 1424-1432; Iuchi, T., et al., 2008. Cancer Res 68: 4431-4441). In contrast, B cells are often overlooked in tumor immunology, likely because of the common notion that humoral and cytolytic responses work in opposition. In previous studies, B cell function in host immune responses was mainly focused on antigen presentation and antibody production. Nevertheless, recent advances in B cell biology have capitalized on old findings and demonstrated that B cells can act either as effector cells (Li, Q., et al., 2009. J Immunol 183: 3195-3203; Li, Q., et al., 2011. Clin Cancer Res 17: 4987-4995) or as regulatory cells (Mizoguchi, A., and A. K. Bhan. 2006. J Immunol 176: 705-710; Mauri, C., and M. R. Ehrenstein. 2008. Trends Immunol 29: 34-40).

The roles of B cells in tumor immunology have demonstrated significant diversity. B cells are phenotypically and functionally heterogeneous (Lapointe, R., et al., 2003. Cancer Res 63: 2836-2843; Lundy, S. K. 2009. Inflamm Res 58: 345-357), and play multiple roles in tumor immunity. On one hand, in vivo primed and in vitro activated B cells have shown efficacy in adoptive immunotherapy of cancer (Li, Q., et al., 2009. J Immunol 183: 3195-3203; Li, Q., et al., 2011. Clin Cancer Res 17: 4987-4995), and the effector B cells can directly kill tumor cells in the absence of antibody (Li, Q., et al., 2011. Clin Cancer Res 17: 4987-4995). On the other hand, resting B cells can promote the development or progression of cancer (Perricone, M. A., et al., 2004. J Immunother 27: 273-281; Qin, Z., et al., 1998. Nat Med 4: 627-630; Shah, S., A. et al., 2005. Int J Cancer 117: 574-586; Evans, D. E., et al., 2000. J Immunol 164: 688-697).

Additional methods of cancer immunotherapy are needed.

### SUMMARY OF THE INVENTION

The present invention relates to compositions and methods for cancer immunotherapy. In particular, the present invention relates to engineered effector B cells and their use in cancer immunotherapy.

Embodiments of the present invention provide uses and methods of treating cancer, comprising: a) isolating B cells from a subject diagnosed with cancer; b) engineering the B cells ex vivo to express high levels of FasL (e.g., GenBank Accession No. U11821) and/or CXCR4 (e.g., GenBank Accession No. AY242129); c) optionally activating the B cells (e.g., with lipopolysaccharide (LPS) and anti-CD40 monoclonal antibody); and d) administering said engineered and activated B cells to the subject. In some embodiments, the cell overexpresses the FasL and/or CXCR4. In some embodiments, the FasL and/or CXCR4 genes are operably linked to a non-native or exogenous promoter. In some embodiments, the treating comprises a method selected from genetic modification (e.g., knock-in of the FasL and/or CXCR4 genes), or nucleic acid treatment (e.g., siRNA, antisense, miRNA, or shRNA treatment to alter expression of FasL and/or CXCR4 repressors or regulators). In some embodiments, B cells are further engineered to reduce or eliminate expression of IL-10. In some embodiments, B cells are isolated from tumor draining lymph nodes, blood, or splenocytes. In some embodiments, the B cells are CD19+ B cells. In some embodiments, 1 million to 100 million (e.g., 1 million to 5 million) engineered B cells are administered to the subject.

In some embodiments, the administering step further comprises administering an anti-IL-10 antibody to the subject. In some embodiments, the method further comprises the step of isolating T-cells from the subject, activating the T cells ex vivo to generate effector T cells, and administering the activated T cells to the subject. In some embodiments, the T cells are activated with anti-CD3 and anti-CD28 monoclonal antibodies and expanded in IL-2.

In some embodiments, the method further comprises the administration of one or more additional cancer therapies (e.g., chemotherapy, radiation therapy, surgery, or immunological therapies).

In some embodiments, the present invention provides a composition (e.g., a pharmaceutical composition), comprising B cells engineered to express exogenous FasL and/or CXCR4. In some embodiments, the pharmaceutical composition further comprises activated T cells. In some embodiments, the engineered B cells lack a function IL-10 gene.

Additional embodiments provide the use of the aforementioned engineered B cells in the treatment of cancer or in the preparation of a medicament for the treatment of cancer.

Additional embodiments are described herein.

### DESCRIPTION OF THE DRAWINGS

FIG. 1 shows the phenotype of 4T1 TDLN B cells and healthy B cells. Detection of IL-10-producing cells in (A, D) WT and (B, E) IL-10^{-/-} CD19⁺ B cells purified from 4T1 TDLNs. (C, F) Detection of IL-10-producing cells in CD19+ B cells purified from WT healthy LNs.

FIG. 2 shows that IL-10^{-/-} 4T1 TDLN B cells are more effective than WT 4T1 TDLN B cells in vitro and in vivo. (A) Number of pulmonary metastatic nodules after adoptive transfer of WT versus IL-10^{-/-} B cells. (B) Cytotoxicity of 4T1 tumor cells by activated WT versus IL-10^{-/-} 4T1 TDLN B cells as measured in an LDH release assay.

FIG. 3 shows the effect of IL-10 neutralization on the antitumor reactivity of adoptively transferred WT 4T1 TDLN B cells. (A) B cells were adoptively transferred with or without IL-10 antibody administration in mice with intramammary fat pad tumors. (B) Antitumor reactivity of IL-10 antibody alone.

FIG. 4 shows the effect of IL-10 neutralization on the cytotoxicity of 4T1 tumor cells. (A) purified and activated T cells, (B) B cells from PBMCs; or (C) T cells, (D) B cells from spleens.

FIG. 5 shows the effect of anti-FasL blockade on the cytotoxicity of 4T1 TDLN B cells against 4T1 tumor cells. (A) B cells were cocultured with 4T1 tumor cells with or without the addition of anti-FasL mAb at 10 or 30 ng/mL. (B) Detection of FasL in B cells purified from

WT and IL-10^{-/-} 4T1 TDLNs after A/E and cocultured with 4T1 tumor cells. (C) Detection of Fas on 4T1 tumor cells.

FIG. 6 shows trafficking of activated TDLN B cells in tumor-bearing and healthy mice.

(A) Phase contrast and fluorescence microscopy of CMTMR-stained B cells are shown. (C) Comparison of detected labeled B cells in lungs of tumor-bearing mice and healthy mice.

### DEFINITIONS

As used herein, the term “subject” refers to any animal (e.g., a mammal), including, but not limited to, humans, non-human primates, rodents, and the like (e.g., which is to be the recipient of a particular treatment, or from whom B-cells are harvested). Typically, the terms “subject” and “patient” are used interchangeably, unless indicated otherwise herein.

As used herein, the term “subject is suspected of having cancer” refers to a subject that presents one or more signs or symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, and blood test. A “preliminary diagnosis” is one based only on visual (e.g., CT scan or the presence of a lump) and/or molecular tests.

As used herein, the term “effective amount” refers to the amount of a composition or treatment sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment to a subject. Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

“Co-administration” refers to administration of more than one chemical agent or therapeutic treatment (e.g., radiation therapy) to a physiological system (e.g., a subject or in vivo, in vitro, or ex vivo cells, tissues, and organs). “Co-administration” of the respective chemical agents and therapeutic treatments (e.g., radiation therapy) may be concurrent, or in any temporal order or physical combination. In some embodiments, “co-administration” refers to adoptive transfer of effector T cells and B cells.

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered. It is intended that the terms “drug” and “chemotherapeutic agent” encompass anti-hyperproliferative and antineoplastic compounds as well as other biologically therapeutic compounds. Examples of drugs are found in Table 1 below.

As used herein, the terms “interfering RNA” and “interfering RNA molecule” refer to all RNA or RNA-like molecules that can interact with RISC and participate in RISC-mediated changes in gene expression. Examples of other interfering RNA molecules that can interact with RISC include, but are not limited to, short hairpin RNAs (shRNAs), single-stranded siRNAs, microRNAs (miRNAs), picoRNAs (piRNAs), and dicer-substrate 27-mer duplexes. Examples of “RNA-like” molecules that can interact with RISC include siRNA, single-stranded siRNA, miRNA, piRNA, and shRNA molecules that contain one or more chemically modified nucleotides, one or more non-nucleotides, one or more deoxyribonucleotides, and/or one or more non-phosphodiester linkages. Thus, siRNAs, single-stranded siRNAs, shRNAs, miRNAs, piRNA, and dicer-substrate 27-mer duplexes are subsets of “interfering RNAs” or “interfering RNA molecules.”

As used herein, “antisense compound” means a compound comprising or consisting of an oligonucleotide at least a portion of which is complementary to a target nucleic acid to which it is capable of hybridizing, resulting in at least 5 one antisense activity.

As used herein, “antisense activity” means any detectable and/or measurable change attributable to the hybridization of an antisense compound to its target nucleic acid.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

### DETAILED DESCRIPTION OF THE INVENTION

The present invention relates to compositions and methods for cancer immunotherapy. In particular, the present invention relates to engineered effector B cells and their use in cancer immunotherapy.

It was previously demonstrated that about 40% of the tumor-draining lymph node (TDLN) cells are CD19⁺ B cells. In vivo sensitized and in vitro activated B cells could mediate tumor regression in cancer adoptive immunotherapy (7, 8). TDLN B cells secondarily activated with LPS and anti-CD40 resulted in the generation of therapeutic effector B cells (>95% CD19⁺ cells), and adoptive transfer of these activated TDLN B cells suppressed tumor growth in tumor-bearing mice (7). Using a murine 4T1 pulmonary metastatic model, it was found that adoptive transfer of LPS/anti-CD40-activated 4T1 TDLN B cells significantly inhibited the development of spontaneous 4T1 pulmonary metastasis in tumor-bearing mice, and the 4T1 TDLN B effector cells mediated specific 4T1 tumor cell lysis in vitro in the absence of antibody and other effector cells (8).

In experiments described herein, experimental evidence that the direct killing of tumor cells by activated TDLN B cells involves both Fas/FasL and CXCR4/CXCL12 pathways was obtained. In addition, IL-10 was found to regulate the CTL activity of PBMC T cells as well as splenic T and B cells isolated from hosts after B cell adoptive transfer.

The Fas/FasL axis has shown a key role in T cells during autoimmunity, infections, and cancer (12). LPS-activated B splenocytes and B cells from normal lymphoid tissues express FasL (29, 30, 41). Klinker et al. reported that activated B cells expressed FasL and such B cells induced T cell apoptosis via Fas/FasL axis (50). Experiments described herein investigated the mechanisms by which in vitro LPS/anti-CD40-activated TDLN B cells directly kill tumor cells, and found that FasL blockade using anti-FasL antibody significantly reduced B cell-mediated direct killing of tumor cells, and such effect was anti-FasL dose dependent. In addition, the CXCR4/CXCL12 axis plays an important role in tumor growth and metastasis, and its role in cancer cell-tumor microenvironment interaction has recently been studied regarding the gastric cancer progression and the attraction/activation of leukocytes (32, 51, 52). While it was reported that CXCR4 may be expressed on both lymphocytes and cancer cells (33), it was found that 4T1 TDLN B cells expressed CXCR4, but its expression on 4T1 tumor cells was very low.

Embodiments of the present invention provides compositions, systems, and methods for cancer immunotherapy using ex vivo engineered B cells, alone or in combination with activated T cells.

### I. Engineered B Cells

Embodiments of the present invention provide engineered B cells (e.g., for use in cancer immunotherapy). The present disclosure is not limited to a source of B cells. In some embodiments, B cells for cancer immunotherapy are autologous. In some embodiments, B cells are isolated from a subject diagnosed with cancer.

A source of cells includes, without limitation, blood, blood fraction (e.g., plasma, serum, buffy coat, red blood cell layer), peripheral blood mononuclear cells (PBMC), bone marrow, biological fluid (e.g., urine, blood, saliva, amniotic fluid, exudate from a region of infection or inflammation, mouth wash, cerebral spinal fluid, synovial fluid), or organ, tissue, cell, cell pellet, cell extract or biopsy (e.g., brain, neck, spine, throat, heart, lung, breast, kidney, liver, intestine, colon, pancreas, bladder, cervix, testes, skin and the like). The source can be directly from the patient or donor, sometimes is frozen, and at times is provided as a cell suspension.

Cells from a patient sometimes are from patient blood, and in certain embodiments are immune cells, such as simulator white blood cells or lymphocytes or dendritic cells from the blood. Cells from a donor sometimes are from donor blood, and in certain embodiments are white blood cells or lymphocytes from the blood. Stimulator donor blood and or buffy coat sometimes is from a blood bank. Blood sometimes is peripheral blood, sometimes is a blood fraction (e.g., buffy coat), sometimes is zero to seven days old, and at times is frozen blood or frozen blood fraction (e.g., blood cells are vitally cryopreserved).

In some embodiments, B-cells are isolated from tumor draining lymph nodes (TDLN) (e.g., of a subject diagnosed with cancer). In some embodiments, lymph nodes are isolated, processed (e.g., using mechanical dissociation), filtered, and washed. B-cells are then isolated from the processed TDLN cells.

In some embodiments, B cells are CD19⁺ B cells. In some embodiments, CD19+ B cell are isolated (e.g., from processed TDLN) using anti-CD19 capture (e.g., using a solid support functionalized with ant-CD19 monoclonal antibodies) or other suitable method.

In some embodiments, B cells that do not express IL-10 are isolated and separated from the B cell population (e.g., using flow cytometry).

In some embodiments, B cells are isolated from a patient diagnosed with a cancer. In some embodiments, the patient has undergone a conditioning step (e.g., chemotherapy, radiation, or other cancer therapy).

Following isolation, B cells are engineered to express FasL and/or CXCR4. In some embodiments, B cells are further engineered to repress or eliminate expression of IL-10.

In some embodiments, following or prior to engineering, B cells are activated (e.g., using the methods described herein).

B cells isolated and engineered using the methods described herein find use in a variety of applications. In some therapeutic embodiments, B cells are re-introduced to the subject they were originally isolated from (e.g., to provide cancer immunotherapy).

Methods for engineering and activating B cells are described in detail below.

A. Engineering of B Cells

The present disclosure is not limited to a method of isolating and engineering B cells. In some embodiments, B cells are engineered to express Fas/FasL and/or CXCR4/CXCL12 pathway genes (e.g., FasL and/or CXCR4). In some embodiments, B cells are further engineered to reduce or eliminate expression of IL-10.

Examples of techniques for engineering B cells to express FasL and/or CXCR4 (and optionally to decrease expression of IL-10) include, but are not limited to, genetic methods (e.g., gene knock-in or knock-out) and nucleic acid based methods (e.g., antisense, miRNA, siRNA, and shRNA).

1. Genetic Methods

In some embodiments, genetic methods are used to introduce expression of FasL and/or CXCR4. Examples of genetic manipulation include, but are not limited to gene addition (e.g., “knock-in” of FasL and/or CXCR4 genes) and gene knockout (e.g., removing the IL-10 gene from the chromosome using, for example, recombination).

In some embodiments, gene knock-in methods utilize introduction of nucleic acids encoding FasL and/or CXCR4 into B cells ex vivo.

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. In some embodiments, delivery of naked DNA utilizes organically modified silica or silicate (ormosil).

In some embodiments, methods use gene delivery vehicles derived from viruses, including, but not limited to, adenoviruses, retroviruses, vaccinia viruses, and adeno-associated viruses.

Retroviruses are one of the mainstays of current gene therapy approaches. The recombinant retroviruses such as the Moloney murine leukemia virus have the ability to integrate into the host genome in a stable fashion. They contain a reverse transcriptase that allows integration into the host genome.

Retroviral vectors can either be replication-competent or replication-defective. Replication-defective vectors are the most common choice in studies because the viruses have had the coding regions for the genes necessary for additional rounds of virion replication and packaging replaced with other genes, or deleted. These viruses are capable of infecting their target cells and delivering their viral payload, but then fail to continue the typical lytic pathway that leads to cell lysis and death.

Conversely, replication-competent viral vectors contain all necessary genes for virion synthesis, and continue to propagate themselves once infection occurs. Because the viral genome for these vectors is much lengthier, the length of the actual inserted gene of interest is limited compared to the possible length of the insert for replication-defective vectors. Depending on the viral vector, the typical maximum length of an allowable DNA insert in a replication-defective viral vector is usually about 8-10 kB. While this limits the introduction of many genomic sequences, most cDNA sequences can still be accommodated.

Lentiviruses are a subclass of Retroviruses. They have recently been adapted as gene delivery vehicles (vectors) thanks to their ability to integrate into the genome of non-dividing cells, which is the unique feature of Lentiviruses as other Retroviruses can infect only dividing cells. The viral genome in the form of RNA is reverse-transcribed when the virus enters the cell to produce DNA, which is then inserted into the genome at a random position by the viral integrase enzyme. The vector, now called a provirus, remains in the genome and is passed on to the progeny of the cell when it divides. The site of integration is unpredictable, which can pose a problem. The provirus can disturb the function of cellular genes and lead to activation of oncogenes promoting the development of cancer, which raises concerns for possible applications of lentiviruses in gene therapy. However, studies have shown that lentivirus vectors have a lower tendency to integrate in places that potentially cause cancer than gamma-retroviral vectors.

For safety reasons lentiviral vectors never carry the genes required for their replication. To produce a lentivirus, several plasmids are transfected into a so-called packaging cell line, commonly HEK 293. One or more plasmids, generally referred to as packaging plasmids, encode the virion proteins, such as the capsid and the reverse transcriptase. Another plasmid contains the genetic material to be delivered by the vector. It is transcribed to produce the single-stranded RNA viral genome and is marked by the presence of the ψ (psi) sequence. This sequence is used to package the genome into the virion.

Adeno-associated virus (AAV) is a small virus that infects humans and some other primate species. AAV is not currently known to cause disease and consequently the virus causes a very mild immune response. AAV can infect both dividing and non-dividing cells and may incorporate its genome into that of the host cell. These features make AAV a very attractive candidate for creating viral vectors for gene therapy.

Furthermore, because of its potential use as a gene therapy vector, researchers have created an altered AAV called Self-complementary adeno-associated virus (scAAV). Whereas AAV packages a single strand of DNA and requires the process of second-strand synthesis, scAAV packages both strands which anneal together to form double stranded DNA. By skipping second strand synthesis scAAV allows for rapid expression in the cell (McCarty, D M; Monahan, P E; Samulski, R J (2001). “Self-complementary recombinant adeno-associated virus (scAAV) vectors promote efficient transduction independently of DNA synthesis”. Gene Therapy 8 (16): 1248-54). Otherwise, scAAV carries many characteristics of its AAV counterpart.

Because of the higher efficiency as compared to retroviruses, vectors derived from adenoviruses are the preferred gene delivery vehicles for transferring nucleic acid molecules into host cells in vivo. The adenoviruses (Ads) comprise a large family of double-stranded DNA viruses found in amphibians, avians, and mammals which have a nonenveloped icosahedral capsid structure (Straus, Adenovirus infections in humans. In The Adenoviruses. 451-498, 1984; Hierholzer et al., J. Infect. Dis., 158: 804-813, 1988; Schnurr and Dondero, Intervirology., 36: 79-83, 1993; Jong et al., J Clin Microbiol., 37:3940-3945:1999). In contrast to retroviruses, adenoviruses can transduce numerous cell types of several mammalian species, including both dividing and nondividing cells, without integrating into the genome of the host cell.

Generally speaking, adenoviral DNA is typically very stable and remains episomal (e.g., extrachromosomal), unless transformation or tumorigenesis has occurred. In addition, adenoviral vectors can be propagated to high yields in well-defined production systems which are readily amenable to pharmaceutical scale production of clinical grade compositions. Typically, the production of recombinant adenoviral vectors relies on the use of a packaging cell line which is capable of complementing the functions of adenoviral gene products that have been either deleted or engineered to be nonfunctional.

Presently, two well-characterized human subgroup C adenovirus serotypes (i.e., hAd2 and hAd5) are widely used as the sources of the viral backbone for most of the adenoviral vectors that are used for genetic therapy. Replication-defective human adenoviral vectors have also been used. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

2. RNA Interference (RNAi)

In some embodiments, RNAi is utilized to inhibit upstream or downstream regulators of FasL and/or CXCR4 expression (e.g., to reduce expression of FasL and/or CXCR4 repressors) and optionally to inhibit IL-10 expression in B cells.

RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Comers, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol Biol. 2005 May 13; 348(4):883-93, J Mol Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

In some embodiments, siRNA treatment further encompasses micro-RNAs (miRNA), functional small-hairpin RNA (shRNA), or other dsRNAs which can be expressed in vivo using DNA templates with RNA polymerase III promoters (Zeng et al., Mol. Cell. 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Sui et al., Proc. Natl. Acad. Sci. USA 99(6):5515-5520 (2002)), each of which are incorporated herein by reference in their entirety.

In some embodiments, IL-10 expression is inhibited using small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′UU-overhangs; subsequently, the ends of these shRNAs are processed, converting the shRNAs into siRNA-like molecules of about 21 nucleotides (Brummelkamp et al., Science 296:550-553 (2002); Miyagishi and Taira, Nature Biotechnol. 20:497-500 (2002).

In some embodiments, IL-10 expression is inhibited using miRNA treatment. Animal cells express a range of noncoding RNAs of approximately 22 nucleotides termed micro RNA (miRNAs) which can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells. When expressed by DNA vectors containing polymerase III promoters, micro-RNA designed hairpins can silence gene expression.

3. Antisense

In some embodiments, antisense is utilized to inhibit upstream or downstream regulators of FasL and/or CXCR4 expression (e.g., to reduce expression of FasL and/or CXCR4 repressors) and optionally to inhibit IL-10 expression in B cells.

The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of cancer markers of the present invention. In the context of the present invention, “modulation” means either an increase (stimulation) or a decrease (inhibition) in the expression of a gene. For example, expression may be inhibited to potentially prevent tumor proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a cancer marker of the present invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Publ. No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

B. Activation of B Cells

In some embodiments, prior to or following ex vivo engineering, B cells are activated ex vivo. The present disclosure is not limited to a particular method of activation. In some embodiments, B cells are activated (e.g., with LPS plus anti-CD40 mAb) in medium (e.g., complete medium (CM)) containing human recombinant IL-2 (See e.g., Qiao Li et al., The Journal of Immunology, 2009, 183: 3195-3203 and Qiao Li, et al, Clin Cancer Res 2011; 17:4987-4995).

C. T Cell Preparation

In some embodiments, T cells are isolated from a subject, activated ex vivo, and administered in combination with engineered B cells. T cells are isolated from any suitable source (e.g., those described above). In some embodiments, T cells are isolated from peripheral blood mononuclear cells (PBMC) or splenocytes. In some embodiments, T cells are CD3+ and are isolated using CD3 capture methods (e.g., solid supports functionalized with CD3 monoclonal antibodies) or other suitable methods.

The present disclosure is not limited to a particular method of activating T cells for use in cancer immunotherapy. In some embodiments, T cells are activated with immobilized anti-CD3 and anti-CD28 mAbs in media containing IL-2 as described in (7, 8).

D. Pharmaceutical Compositions

Engineered B cells (e.g., alone or in combination with activated T cells) may be formulated in a pharmaceutical composition in any manner appropriate for administration to a subject. A composition may be prepared by washing cells one or more times with a medium compatible with cells of the subject (e.g., phosphate buffered saline). Cells also may be combined with components that form a time-release matrix or gel in some embodiments. Non-limiting examples of components that form a matrix include, without limitation, fibrin, proteoglycans or polysaccharides. A matrix sometimes is a thrombus or plasma clot in some embodiments.

A composition can be administered to a subject in need thereof in amount effective to treat a cell proliferative condition (e.g., cancer, tumor), inflammation condition or autoimmune condition. The terms “treat” and “treating” as used herein refer to (i) preventing a disease or condition from occurring (e.g. prophylaxis); (ii) inhibiting the disease or condition or arresting its development; (iii) relieving the disease or condition; and/or (iv) ameliorating, alleviating, lessening, and removing symptoms of the disease or condition. The terms also can refer to reducing or stopping a cell proliferation rate (e.g., slowing or halting tumor growth) or reducing the number of proliferating cancer cells (e.g., removing part or all of a tumor).

In some embodiments, Engineered B cells (e.g., alone or in combination with activated T cells) are administered to a part of the body that does not rapidly inactivate the administered B cells. In certain embodiments, activated B cells can be administered to an immuno-privileged region of a subject. An immuno-privileged region sometimes is characterized by one or more of the following non-limiting features: low expression of MHC molecules; increased expression of surface molecules that inhibit complement activation; local production of immunosuppressive cytokines such as TGF-beta; and presence of neuropeptides. An immuno-privileged region can be semi-immuno-privileged, where a minority subset of cells are subject to the immune system. In certain embodiments, a composition is administered to the brain, an immuno-privileged region, to treat a cancer, where cancer cells are the predominant antigen presenting cells and are preferentially killed by the B cells over non-cancer cells. Other non-limiting examples of immuno-privileged regions of the body are portions of the eye (e.g., ocular anterior chamber, ocular uveal tract, cornea, central nervous system), testis, liver and pregnant uterus.

In some embodiments, engineered B cells (e.g., alone or in combination with activated T cells) are administered to another part of the body that is not immuno-privileged, in certain embodiments. In some embodiments, activated B cells are administered to a part of the body where B cells are not substantially cleared or inactivated. For example, activated B cells may be administered directly to a solid tumor mass, where the B cells may not be readily transported to other parts of the body or inactivated (e.g., injected into the tumor). Compositions can be administered to the subject at a site of a tumor, in some embodiments. Diffuse cancers are treatable where the composition is maintained in contact with cells within a limited area (e.g., within the cranial cavity), in certain embodiments.

In some embodiments, engineered B cells (e.g., alone or in combination with activated T cells) are delivered in any suitable manner. A dose can be administered by any suitable method, including, but not limited to, systemic administration, intratumoral administration, bolus injection, infusion, convection enhanced delivery, blood-brain barrier disruption, intracarotid injection, implant delivery (e.g., cytoimplant), and combinations thereof (e.g., blood-brain barrier disruption followed by intracarotid injection). Blood-brain barrier disruption can include, without limitation, osmotic disruption; use of vasoactive substances (e.g., bradykinin); exposure to high intensity focused ultrasound (HIFU); use of endogenous transport systems, including carrier-mediated transporters such as glucose and amino acid carriers, for example; receptor-mediated transcytosis for insulin or transferrin; blocking of active efflux transporters such as p-glycoprotein, for example; intracerebral implantation; convection-enhanced distribution; use of a liposome; and combinations of the foregoing. Engineered B cells are delivered by injection in a suitable volume (e.g., about 5 ml to about 20 ml volume (e.g., about 10 ml volume)), and in a suitable medium (e.g., saline; phosphate buffered saline). An implant sometimes includes a gel or matrix. In certain embodiments, an infusion is via a catheter and/or reservoir (e.g., Rickham, Ommaya reservoir).

The dose given is an amount “effective” in bringing about a desired therapeutic response (e.g., destruction of cancer cells). For pharmaceutical compositions described herein, an effective dose often falls within the range of about 1 to 500 million (e.g., 1 to 20 million, 1 to 10 million, or 1 to 5 million) cells.

### II. Treatment Methods

In some embodiments, the present disclosure provides compositions and methods for treating cancer using engineered B cells (e.g., alone or in combination with activated T cells). In some embodiments, multiple doses are delivered over time to achieve a desired effect, and often, each dose delivers an effective amount of cells. For example, in some embodiments, Engineered B cells are administered daily, weekly, monthly, annually, or less often. In some embodiments, treatment is stopped after a period of time and re-started at a later date (e.g., if a cancer has recurred or as maintenance therapy).

In some embodiments, Engineered B cells are administered in combination with an antibody that specifically binds to IL-10 (e.g., to render the B cells IL-10^{-1/1}) Suitable antibodies include, but are not limited to, those disclosed herein.

In some embodiments, methods and compositions provided herein are utilized to treat a cell proliferative condition. Examples of cell proliferation disorders, include, without limitation, cancers of the colorectum, breast, lung, liver, pancreas, lymph node, colon, prostate, brain, head and neck, skin, liver, kidney, and heart. Examples of cancers include hematopoietic neoplastic disorders, which are diseases involving hyperplastic/neoplastic cells of hematopoietic origin (e.g., arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (reviewed in Vaickus, Crit. Rev. in Oncol./Hemotol. 11:267-297 (1991)); lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. In a particular embodiment, a cell proliferative disorder is non-endocrine tumor or endocrine tumors. Illustrative examples of non-endocrine tumors include but are not limited to adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor may be an islet cell tumor. Also included are pancreatic tumors (e.g., as pancreatic ductal adenocarcinomas); lung tumors (e.g., small and large cell adenocarcinomas, squamous cell carcinoma, and bronchoalveolar carcinoma); colon tumors (e.g., epithelial adenocarcinoma, and liver metastases of these tumors); liver tumors (e.g., hepatoma, cholangiocarcinoma); breast tumors (e.g., ductal and lobular adenocarcinoma); gynecologic tumors (e.g., squamous and adenocarcinoma of the uterine cervix, anal uterine and ovarian epithelial adenocaroinoma); prostate tumors (e.g., prostatic adenocarcinoma); bladder tumors (e.g., transitional, squamous cell carcinoma); tumors of the reticuloendothelial system (RES) (e.g., B and T cell lymphoma (nodular and diffuse), plasmacytoma and acute and chronic leukemia); skin tumors (e.g., malignant melanoma); and soft tissue tumors (e.g., soft tissue sarcoma and leiomyosarcoma).

A cell proliferation disorder may be a tumor in an immune-privileged site, such as the brain, for example. A brain tumor is an abnormal growth of cells within the brain or inside the skull, which can be cancerous or non-cancerous (benign). A brain tumor is any intracranial tumor having (and/or arising from) abnormal and uncontrolled cell division, often in the brain itself (neurons, glial cells (astrocytes, oligodendrocytes, ependymal cells), lymphatic tissue, blood vessels), in the cranial nerves (myelin-producing Schwann cells), in the brain envelopes (meninges), skull, pituitary and pineal gland, or spread from cancers primarily located in other organs (metastatic tumors). Primary brain tumors sometimes are located infratentorially in the posterior cranial fossa (often in children) and in the anterior two-thirds of the cerebral hemispheres or supratentorial location (often in adults), although they can affect any part of the brain. Non-limiting types of brain tumors include glioma (e.g., mixed glioma), glioblastoma (e.g., glioblastoma multiforme), astrocytoma (e.g., anaplastic astrocytoma), oligodendroglioma, medulloblastoma, ependymoma, brain stem tumors, primitive neural ectodermal tumor, and pineal region tumors.

A pharmaceutical composition provided herein may be administered following, preceding, in lieu of, or in combination with, one or more other therapies relating to generating an immune response or treating a condition in the subject (e.g., cancer). For example, the subject may previously or concurrently be treated by chemotherapy, radiation therapy, surgery, cell therapy and/or a forms of immunotherapy and adoptive transfer. Where such modalities are used, they often are employed in a way or at a time that does not interfere with the immunogenicity of compositions described herein. The subject also may have been administered another vaccine or other composition to stimulate an immune response. Such alternative compositions may include tumor antigen vaccines, nucleic acid vaccines encoding tumor antigens, anti-idiotype vaccines, and other types of cellular vaccines, including cytokine-expressing tumor cell lines. Non-limiting examples of chemotherapeutic agents include, without limitation, alkylating agents (e.g., cisplatin); antimetabolites (e.g., purine, pyrimidine); plant alkaloids and terpenoids (e.g., taxanes); vinca alkaloids and topoisomerase inhibitors. Surgeries sometimes are tumor removal or cytoreduction, the latter of which is removal of as much tumor as possible to reduce the number of tumor cells available for proliferation. Surgeries include, without limitation, surgery through the nasal cavity (trans-nasal), surgery through the skull base (trans-sphenoidal), and craniotomy (opening of the skull). Radiotherapies include, without limitation, external beam radiotherapy (EBRT or XBRT) or teletherapy, brachytherapy or sealed source radiotherapy, systemic radioisotope therapy or unsealed source radiotherapy, virtual simulation, 3-dimensional conformal radiotherapy, intensity-modulated radiotherapy, particle therapy and radioisotope therapy. Conventional external beam radiotherapy (2DXRT) often is delivered via two-dimensional beams using linear accelerator machines. Stereotactic radiotherapy is a type of external beam radiotherapy that focuses high doses of radiation within the body (e.g., cyberknife, gamma knife and Novalis Tx). Cell therapies include, without limitation, administration alone or in combination of dendritic cells, alloreactive cytotoxic T-lymphocytes, stem cells, and monocytes.

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use with the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; and 23) radiation.

Any oncolytic agent used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., Emeryville, CA |
| (des-alanyl-1, serine-125 human interleukin-2) | | |
| Alemtuzumab | Campath | Millennium and ILEX |
| (IgG1κ anti CD52 antibody) | | Partners, LP, Cambridge, MA |
| Alitretinoin | Panretin | Ligand Pharmaceuticals, Inc., |
| (9-cis-retinoic acid) | | San Diego CA |
| Allopurinol | Zyloprim | GlaxoSmithKline, Research |
| (1,5-dihydro-4 H-pyrazolo[3,4-d]pyrimidin-4-one | | Triangle Park, NC |
| monosodium salt) | | |
| Altretamine | Hexalen | US Bioscience, West |
| (N,N,N′,N′,N″,N″,-hexamethyl-1,3,5-triazine-2,4,6- | | Conshohocken, PA |
| triamine) | | |
| Amifostine | Ethyol | US Bioscience |
| (ethanethiol, 2-[(3-aminopropyl)amino]-, dihydrogen | | |
| phosphate (ester)) | | |
| Anastrozole | Arimidex | AstraZeneca Pharmaceuticals, |
| (1,3-Benzenediacetonitrile, a,a,a′,a′-tetramethyl-5-(1H- | | LP, Wilmington, DE |
| 1,2,4-triazol-1-ylmethyl)) | | |
| Arsenic trioxide | Trisenox | Cell Therapeutic, Inc., Seattle, |
| | | WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, Corp., |
| (lyophilized preparation of an attenuated strain of | | Durham, NC |
| Mycobacterium bovis (Bacillus Calmette-Gukin [BCG], | | |
| substrain Montreal) | | |
| bexarotene capsules | Targretin | Ligand Pharmaceuticals |
| (4-[1-(5,6,7,8-tetrahydro-3,5,5,8,8-pentamethyl-2- | | |
| napthalenyl) ethenyl] benzoic acid) | | |
| bexarotene gel | Targretin | Ligand Pharmaceuticals |
| Bleomycin | Blenoxane | Bristol-Myers Squibb Co., |
| (cytotoxic glycopeptide antibiotics produced by | | NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]-cytidine) | | |
| Carboplatin | Paraplatin | Bristol-Myers Squibb |
| (platinum, diammine [1,1-cyclobutanedicarboxylato(2-)- | | |
| 0,0′]-, (SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford Pharmaceuticals, |
| | | Inc., Baltimore, MD |
| Celecoxib | Celebrex | Searle Pharmaceuticals, |
| (as 4-[5-(4-methylphenyl)-3-(trifluoromethyl)-1H- | | England |
| pyrazol-1-yl] benzenesulfonamide) | | |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2-CdA | R. W. Johnson Pharmaceutical |
| (2-chloro-2′-deoxy-b-D-adenosine) | | Research Institute, Raritan, NJ |
| Cyclophosphamide | Cytoxan, Neosar | Bristol-Myers Squibb |
| (2-[bis(2-chloroethyl)amino] tetrahydro-2H-13,2- | | |
| oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, Inc., |
| | | San Diego, CA |
| Dacarbazine | DTIC-Dome | Bayer AG, Leverkusen, |
| (5-(3,3-dimethyl-1-triazeno)-imidazole-4-carboxamide | | Germany |
| (DTIC)) | | |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., Thousand Oaks, |
| (recombinant peptide) | | CA |
| daunorubicin liposomal | DanuoXome | Nexstar Pharmaceuticals, Inc., |
| ((8S-cis)-8-acetyl-10-[(3-amino-2,3,6-trideoxy-á-L-lyxo- | | Boulder, CO |
| hexopyranosyl)oxy]-7,8,9,10-tetrahydro-6,8,11- | | |
| trihydroxy-1-methoxy-5,12-naphthacenedione | | |
| hydrochloride) | | |
| Daunorubicin HCl, daunomycin | Cerubidine | Wyeth Ayerst, Madison, NJ |
| ((1S,3S)-3-Acetyl-1,2,3,4,6,11-hexahydro-3,5,12- | | |
| trihydroxy-10-methoxy-6,11-dioxo-1-naphthacenyl 3- | | |
| amino-2,3,6-trideoxy-(alpha)-L-lyxo-hexopyranoside | | |
| hydrochloride) | | |
| Denileukin diftitox | Ontak | Seragen, Inc., Hopkinton, MA |
| (recombinant peptide) | | |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis Pharmaceuticals, Inc., |
| ((2R,3S)-N-carboxy-3-phenylisoserine, N-tert-butyl ester, | | Bridgewater, NJ |
| 13-ester with 5b-20-epoxy-12a,4,7b,10b,13a- | | |
| hexahydroxytax-11-en-9-one 4-acetate 2-benzoate, | | |
| trihydrate) | | |
| Doxorubicin HCl | Adriamycin, Rubex | Pharmacia & Upjohn |
| (8S,10S)-10-[(3-amino-2,3,6-trideoxy-a-L-lyxo- | | Company |
| hexopyranosyl)oxy]-8-glycolyl-7,8,9,10-tetrahydro- | | |
| 6,8,11-trihydroxy-1-methoxy-5,12-naphthacenedione | | |
| hydrochloride) | | |
| doxorubicin | Adriamycin PFS | Pharmacia & Upjohn |
| | Intravenous injection | Company |
| doxorubicin liposomal | Doxil | Sequus Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one propionate) | | Indianapolis, IN |
| dromostanolone propionate | Masterone injection | Syntex, Corp., Palo Alto, CA |
| Elliott's B Solution | Elliott's B Solution | Orphan Medical, Inc |
| Epirubicin | Ellence | Pharmacia & Upjohn |
| ((8S-cis)-10-[(3-amino-2,3,6-trideoxy-a-L-arabino- | | Company |
| hexopyranosyl)oxy]-7,8,9,10-tetrahydro-6,8,11- | | |
| trihydroxy-8-(hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3-[bis(2- | | Company |
| chloroethyl)carbamate] 17-(dihydrogen phosphate), | | |
| disodium salt, monohydrate, or estradiol 3-[bis(2- | | |
| chloroethyl)carbamate] 17-(dihydrogen phosphate), | | |
| disodium salt, monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′-(dihydrogen | | |
| phosphate)) | | |
| etoposide, VP-16 | Vepesid | Bristol-Myers Squibb |
| (4′-demethylepipodophyllotoxin 9-[4,6-0-(R)-ethylidene- | | |
| (beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, Inc., |
| (fluorinated nucleotide analog of the antiviral agent | | Cedar Knolls, NJ |
| vidarabine, 9-b-D-arabinofuranosyladenine (ara-A)) | | |
| Fluorouracil, 5-FU | Adrucil | ICN Pharmaceuticals, Inc., |
| (5-fluoro-2,4(1H,3H)-pyrimidinedione) | | Humacao, Puerto Rico |
| Fulvestrant | Faslodex | IPR Pharmaceuticals, |
| (7-alpha-[9-(4,4,5,5,5-penta fluoropentylsulphinyl) | | Guayama, Puerto Rico |
| nonyl]estra-1,3,5-(10)-triene-3,17-beta-diol) | | |
| Gemcitabine | Gemzar | Eli Lilly |
| (2′-deoxy-2′,2′-difluorocytidine monohydrochloride (b- | | |
| isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca Pharmaceuticals |
| (acetate salt of [D-Ser(But)⁶, Azgly¹⁰]LHRH; pyro-Glu- | | |
| His-Trp-Ser-Tyr-D-Ser(But)-Leu-Arg-Pro-Azgly-NH2 | | |
| acetate [C₅₉H₈₄N₁₈O₁₄•(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea covalent | | Cambridge MA |
| bond between the monoclonal antibody Ibritumomab and | | |
| the linker-chelator tiuxetan [N-[2- | | |
| bis(carboxymethyl)amino]-3-(p-isothiocyanatophenyl)- | | |
| propyl]-[N-[2-bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3-amino-2,3,6- | | Company |
| trideoxy-(alpha)-L-lyxo-hexopyranosyl)oxy]-7,8,9,10- | | |
| tetrahydro-6,9,11-trihydroxyhydrochloride, (7S-cis)) | | |
| Ifosfamide | IFEX | Bristol-Myers Squibb |
| (3-(2-chloroethyl)-2-[(2-chloroethyl)amino]tetrahydro- | | |
| 2H-1,3,2-oxazaphosphorine 2-oxide) | | |
| Imatinib Mesilate | Gleevec | Novartis AG, Basel, |
| (4-[(4-Methyl-1-piperazinyl)methyl]-N-[4-methyl-3-[[4- | | Switzerland |
| (3-pyridinyl)-2-pyrimidinyl]amino]-phenyl]benzamide | | |
| methanesulfonate) | | |
| Interferon alfa-2a | Roferon-A | Hoffmann-La Roche, Inc., |
| (recombinant peptide) | | Nutley, NJ |
| Interferon alfa-2b | Intron A (Lyophilized | Schering AG, Berlin, |
| (recombinant peptide) | Betaseron) | Germany |
| Irinotecan HCl | Camptosar | Pharmacia & Upjohn |
| ((4S)-4,11-diethyl-4-hydroxy-9-[(4- | | Company |
| piperi-dinopiperidino)carbonyloxy]-1H-pyrano[3′,4′: | | |
| 6,7] indolizino[1,2-b] quinoline-3,14(4H,12H) | | |
| dione hydrochloride trihydrate) | | |
| Letrozole | Femara | Novartis |
| (4,4′-(1H-1,2,4-Triazol-1-ylmethylene) dibenzonitrile) | | |
| Leucovorin | Wellcovorin, Leucovorin | Immunex, Corp., Seattle, WA |
| (L-Glutamic acid, N[4[[(2amino-5-formyl1,4,5,6,7,8 | | |
| hexahydro4oxo6-pteridinyl)methyl]amino]benzoyl], | | |
| calcium salt (1:1)) | | |
| Levamisole HCl | Ergamisol | Janssen Research Foundation, |
| ((−)-(S)-2,3,5,6-tetrahydro-6-phenylimidazo [2,1-b] | | Titusville, NJ |
| thiazole monohydrochloride C₁₁H₁₂N₂S•HCl) | | |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α(acetyloxy)-6-methylpregna-4,6-diene-3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6 H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L-glutamic | | |
| acid) | | |
| Methoxsalen | Uvadex | Therakos, Inc., Way Exton, Pa |
| (9-methoxy-7H-furo[3,2-g][1]-benzopyran-7-one) | | |
| Mitomycin C | Mutamycin | Bristol-Myers Squibb |
| mitomycin C | Mitozytrex | SuperGen, Inc., Dublin, CA |
| Mitotane | Lysodren | Bristol-Myers Squibb |
| (1,1-dichloro-2-(o-chlorophenyl)-2-(p-chlorophenyl) | | |
| ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10-anthracenedione | | |
| dihydrochloride) | | |
| Nandrolone phenpropionate | Durabolin-50 | Organon, Inc., West Orange, |
| | | NJ |
| Nofetumomab | Verluma | Boehringer Ingelheim Pharma |
| | | KG, Germany |
| Oprelvekin | Neumega | Genetics Institute, Inc., |
| (IL-11) | | Alexandria, VA |
| Oxaliplatin | Eloxatin | Sanofi Synthelabo, Inc., NY, NY |
| (cis-[(1R,2R)-1,2-cyclohexanediamine-N,N′] | | |
| [oxalato(2-)-O,O′] platinum) | | |
| Paclitaxel | TAXOL | Bristol-Myers Squibb |
| (5β,20-Epoxy-1,2a,4,7β,10β,13a-hexahydroxytax-11- | | |
| en-9-one 4,10-diacetate 2-benzoate 13-ester with (2R,3S)- | | |
| N-benzoyl-3-phenylisoserine) | | |
| Pamidronate | Aredia | Novartis |
| (phosphonic acid (3-amino-1-hydroxypropylidene) bis-, | | |
| disodium salt, pentahydrate, (APD)) | | |
| Pegademase | Adagen (Pegademase | Enzon Pharmaceuticals, Inc., |
| ((monomethoxypolyethylene glycol succinimidyl) 11-17- | Bovine) | Bridgewater, NJ |
| adenosine deaminase) | | |
| Pegaspargase | Oncaspar | Enzon |
| (monomethoxypolyethylene glycol succinimidyl | | |
| L-asparaginase) | | |
| Pegfilgrastim | Neulasta | Amgen, Inc |
| (covalent conjugate of recombinant methionyl human G- | | |
| CSF (Filgrastim) and monomethoxypolyethylene glycol) | | |
| Pentostatin | Nipent | Parke-Davis Pharmaceutical |
| | | Co., Rockville, MD |
| Pipobroman | Vercyte | Abbott Laboratories, Abbott |
| | | Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, Inc., |
| | | Vancouver, Canada |
| Procarbazine | Matulane | Sigma Tau Pharmaceuticals, |
| (N-isopropyl-μ-(2-methylhydrazino)-p-toluamide | | Inc., Gaithersburg, MD |
| monohydrochloride) | | |
| Quinacrine | Atabrine | Abbott Labs |
| (6-chloro-9-(1-methyl-4-diethyl-amine) butylamino-2- | | |
| methoxyacridine) | | |
| Rasburicase | Elitek | Sanofi-Synthelabo, Inc., |
| (recombinant peptide) | | |
| Rituximab | Rituxan | Genentech, Inc., South San |
| (recombinant anti-CD20 antibody) | | Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and b)-D- | | |
| glucopyranose and 220 mg citric acid anhydrous) | | |
| Talc | Sclerosol | Bryan, Corp., Woburn, MA |
| (Mg₃Si₄O₁₀ (OH)₂) | | |
| Tamoxifen | Nolvadex | AstraZeneca Pharmaceuticals |
| ((Z)2-[4-(1,2-diphenyl-1-butenyl) phenoxy]-N,N- | | |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as-tetrazine- | | |
| 8-carboxamide) | | |
| teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4-dien-17-oic | | |
| acid [dgr]-lactone) | | |
| Thioguanine, 6-TG | Thioguanine | GlaxoSmithKline |
| (2-amino-1,7-dihydro-6 H-purine-6-thione) | | |
| Thiotepa | Thioplex | Immunex Corporation |
| (Aziridine,1,1′,1″-phosphinothioylidynetris-, or Tris (1- | | |
| aziridinyl) phosphine sulfide) | | |
| Topotecan HCl | Hycamtin | GlaxoSmithKline |
| ((S)-10-[(dimethylamino) methyl]-4-ethyl-4,9-dihydroxy- | | |
| 1H-pyrano[3′,4′: 6,7] indolizino [1,2-b] quinoline-3,14- | | |
| 4H,12H)-dione monohydrochloride) | | |
| Toremifene | Fareston | Roberts Pharmaceutical Corp., |
| (2-(p-[(Z)-4-chloro-1,2-diphenyl-1-butenyl]-phenoxy)- | | Eatontown, NJ |
| N,N-dimethylethylamine citrate (1:1)) | | |
| Tositumomab, I 131 Tositumomab | Bexxar | Corixa Corp., Seattle, WA |
| (recombinant murine immunotherapeutic monoclonal | | |
| IgG₂ₐ lambda anti-CD20 antibody (I 131 is a | | |
| radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA | Vesanoid | Roche |
| (all-trans retinoic acid) | | |
| Uracil Mustard | Uracil Mustard Capsules | Roberts Labs |
| Valrubicin, N-trifluoroacetyladriamycin-14-valerate | Valstar | Anthra --> Medeva |
| ((2S-cis)-2-[1,2,3,4,6,11-hexahydro-2,5,12-trihydroxy-7 | | |
| methoxy-6,11-dioxo-[[4 2,3,6-trideoxy-3- | | |
| [(trifluoroacetyl)-amino-α-L-lyxo-hexopyranosyl]oxyl]-2- | | |
| naphthacenyl]-2-oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′-norvincaleukoblastine [R- | | |
| (R*,R*)-2,3-dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

A composition may be administered in intervals, and may be replenished one or more times. A composition may be administered about 1 to about 20 times. The time interval between each administration independently may be of days or even months, for example 1 month to about 6 months, or about 1 day to about 60 days, or about 1 day to about 7 days. Subsequent administration of a composition described herein can boost immunologic activity and therapeutic activity.

Timing for administering compositions is within the judgment of a managing physician, and depends on the clinical condition of the patient, the objectives of treatment, and concurrent therapies also being administered, for example. Suitable methods of immunological monitoring include a one-way mixed lymphocyte reaction (MLR) using patient lymphoblasts as effectors and tumor cells as target cells. An immunologic reaction also may manifest by a delayed inflammatory response at an injection site or implantation site. Suitable methods of monitoring of a tumor are selected depending on the tumor type and characteristics, and may include CT scan, magnetic resonance imaging (MRI), radioscintigraphy with a suitable imaging agent, monitoring of circulating tumor marker antigens, and the subject's clinical response. Additional doses may be given, such as on a monthly or weekly basis, until the desired effect is achieved. Thereafter, and particularly when an immunological or clinical benefit appears to subside, additional booster or maintenance doses may be administered.

### EXPERIMENTAL

The following example is provided in order to demonstrate and further illustrate certain preferred embodiments and aspects of the present invention and are not to be construed as limiting the scope thereof.

### Example 1

Materials and Methods

Mice

Female wild type (WT) BALB/c mice were purchased from the Jackson Laboratories, Bar Harbor, Me. IL-10 KO (IL-10^{-/-}) mice on BALB/c background are homozygous for a targeted mutation in the IL-10 gene achieved by vectors designed to replace codons 5-55 of exon 1 with a 24 bp linker (providing a termination codon) and a neo expression cassette, to introduce a termination codon into exon 3. They were maintained in a pathogen-free environment and used at age 7 weeks or older. Principles of laboratory animal care (NIH publication No. 85-23, revised 1985) were followed. The animal protocols were approved by the University of Michigan Laboratory of Animal Medicine.

### Murine Tumor Cells

The 4T1 cell line is a mammary carcinoma syngeneic to BALB/c mice (kindly provided by Dr. M. Sabel, University of Michigan). Inoculating 4T1 cells into the mammary fat pad induces the development of spontaneous pulmonary metastases. 4T1 cells were maintained in vitro in complete medium (CM). Renca is a kidney cancer cell line, and TSA is a highly aggressive mammary adenocarcinoma; both are syngeneic to BALB/c mice and were used as specificity controls. Renca and TSA were purchased from American type culture collection (Rockville, Md.). All cell lines were maintained in vitro in complete medium (CM).

### Tumor Draining Lymph Nodes (TDLNs)

In order to induce TDLNs, 1×10⁶ 4 T1 tumor cells in 0.1 ml PBS were injected subcutaneously (s.c.) into the lower flanks of WT or IL-10^{-/-} syngeneic mice. Nine days after 4T1 cell inoculation, the draining inguinal lymph nodes were collected and designated as WT TDLN and IL-10^{-/-} TDLN, respectively. The TDLNs were processed using mechanical dissociation, filtered through nylon mesh and washed in HBSS. Multiple inguinal TDLNs were pooled from groups of mice for lymphoid cell suspension preparation.

### T Cell and B Cell Activation and Expansion

CD19⁺ B cells were purified from the TDLN cells or splenocytes using anti-CD19-coupled microbeads and the MACS separator (MiltenyiBiotec. Inc. Auburn, Calif.). CD3⁺ T cells were purified from the peripheral blood mononuclear cells (PBMC) or splenocytes using anti-CD3-coupled microbeads. B cells were activated with lipopolysaccharide (LPS, Sigma-Aldrich, Atlanta, Ga.) plus anti-CD40 (FGK45) mAb ascites in complete medium (CM) at 37° C. with 5% CO2 for 3-4 days. The anti-CD40 ascites were produced by using FGK45 hybridoma cells (American Type Culture Collection, Rockville, Md.). The use of anti-CD40 mAb ascites at 1/100 dilution was determined by previous titrating tests to be optimal for B cell expansion in combination with LPS (5 μg/ml) (Li, Q., et al., J. Immunol. 2009. 183: 3195-3203; Li, Q., et al., Clin. Cancer Res. 2011. 17: 4987-4995). T cells were activated with immobilized anti-CD3 and anti-CD28 mAbs in CM containing IL-2 as previously described (Li, Q., et al., J. Immunol. 2009. 183: 3195-3203; Li, Q., et al., Clin. Cancer Res. 2011. 17: 4987-4995).

Flow cytometry Analysis

Cell surface expression of CD19, Fas, FasL, and CD25 and intracellular expression of IL-10 were analyzed by immunofluorescence assay. All fluorescein isothiocyanate (FITC)- or phycoerythrin (PE)-conjugated antibodies (FITC or allophycocyanin anti-CD19, PE anti-Fas, PE anti-CD25, PE anti-IL-10, and PE anti-FasL) and matched isotype controls were purchased from BD Biosciences (San Jose, Calif.). To measure intracellular IL-10 expression, 1 million TDLN B cells were incubated with 2 μl/ml Leukocyte Activation Cocktail PMA/ionomycin/Golgiplug, BD Pharmingen, San Jose, Calif.) and 0.67 μl/ml Golgistop (BD Pharmingen) in 6-well plate for 4-6 hours at 37° C. with 5% CO₂. After fixation and permeabilization with Fixation/Perm Buffer (eBioscience, Inc., San Diego, Calif.), the cells were stained with PE rat anti-mouse IL-10 mAb. For FasL expression, purified, and A/E TDLN B cells were cultured with or without 4T1 at 37° C. with 5% CO₂ overnight. After being fixed and permeabilized with Fixation/Perm Buffer (eBioscience, San Diego, Calif.), the cells were stained with anti-FasL. Isotype control staining was used to define the gates for positive and negative cells. Flow cytometry was performed on a LSRII flow cytometer (BD Biosciences). BD FACSDiva software (version 7.0) was used for all flow cytometry analysis.

### Adoptive TDLN B Cell Therapy of 4T1 Cancer

In the first model using IL-10^{-/-} TDLN B cells, healthy BALB/c mice were inoculated with 5×10⁴ 4 T1 cells into the mammary fat pad to induce spontaneous pulmonary metastases. Fourteen days after tumor inoculation, the tumor-bearing mice were treated with tail vein injection of activated WT or IL-10^{-/-} 4T1 TDLN B cells. Commencing on the day of the effector B cell transfer, intraperitoneal (i.p.) injections of IL-2 (40,000 IU) (Novartis, Emeryville, Calif.) were administered in 0.5 ml of PBS and continued twice daily for 8 doses. About 2 weeks after B cell transfer, all mice were sacrificed and lungs were harvested for enumeration of spontaneous pulmonary metastatic nodules as previously described (Li, Q., et al., J. Immunol. 2009. 183: 3195-3203; Li, Q., et al., Clin. Cancer Res. 2011. 17: 4987-4995).

In the second model using anti-IL-10 antibody for IL-10 neutralization, the 4T1 tumor-bearing mice were prepared as in the first model. Fourteen days after tumor inoculation, the mice were treated with tail vein injection of activated WT 4T1 TDLN B cells and IL-2 as in the first model. On the same day of B cell transfer, the tumor-bearing mice were injected with IL-10 or isotype control antibody (Bio X Cell, West Lebanon, N.H.) i.p. at 200 μg per mouse in 0.2 ml PBS daily for 4 days. Approximately 14 days after B cell transfer, all mice were sacrificed, and lungs were harvested for enumeration of spontaneous pulmonary metastatic nodules. At the same time, peripheral blood and spleens were collected for purification of PBMC T cells, splenic T and B cells as described above.

### LDH Cytotoxicity Assay

Cell cytotoxicity was assessed by measuring the release of cytoplasmic lactate dehydrogenase (LDH) into cell culture supernatants according to the manufacturer's protocol (CytoTox 96 Non-Radioactive Cytotoxicity Assay, Promega, Madison, Wis.). For TDLN B cell cytotoxicity, effector B cells were generated from WT or IL-10^{-/-} TDLN B cells using LPS/anti-CD40 as described above. Target cells were plated in triplicates in a 96-well U-bottom tissue culture plate (5000 cells/well) and co-incubated with TDLN B cells at effector to target cell ratios of 1:1, 3:1, 10:1 and 30:1. After 12 hrs of incubation, cells were centrifuged and 50 μl supernatant from each well was transferred to a fresh 96-well plate, 50 IA of the substrate mix was added and incubated at room temperature in the dark for 15 to 30 min. Before LDH measurement, 50 μl of stop solution was added to each well. Maximal release of LDH was performed by incubating the target cells with Lysis Solution (Promega, Madison, Wis.). Target cells without effector cells were used as spontaneous release control. Absorbence was measured at 490 nm using a 96-well plate reader.

For assays of cytotoxicity of effector cells generated from PBMC T or splenic T and B cells, effector T or B cells were generated by anti-CD3/anti-CD28 or LPS/anti-CD40 activation respectively as described above. The lysis assay was performed as described for TDLN B cell cytotoxicity with the exception that T cell killing assay was incubated for 4-6 hours instead of the 12 hours used for B cell-mediated cytotoxicity.

For 4T1 TDLN B cell cytotoxicity in the presence of anti-FasL antibody (Biolegend Inc., San Diego, Calif.) and/or AMD3100 (Sigma, Atlanta, Ga.), the effector B cells were generated as described above, and the experimental process was the same as that of TDLN B cell cytotoxicity with target cells being 4T1 cells, but anti-FasL and/or AMD3100 were added to block FasL and/or CXCR4.

Cytotoxicity was calculated according to the following formula:

$$\% Cytotoxicity = \frac{Experimental \text{-} Effector spontaneous - Target spontaneous}{Target maximum - Target spontaneous}$$

### Statistical Analysis

The significance of differences in numbers of metastatic nodules; the concentrations of cytokine, e.g. IL-10, and cell lysis was determined using one-way analysis of variance (Newman-Keuls post hoc test) or unpaired Student's t-test. P<0.05 was considered statistically significant between the experimental groups.

### Results

IL-10^{-/-} B Cells are More Potent Antitumor Effector Cells than WT B Cells

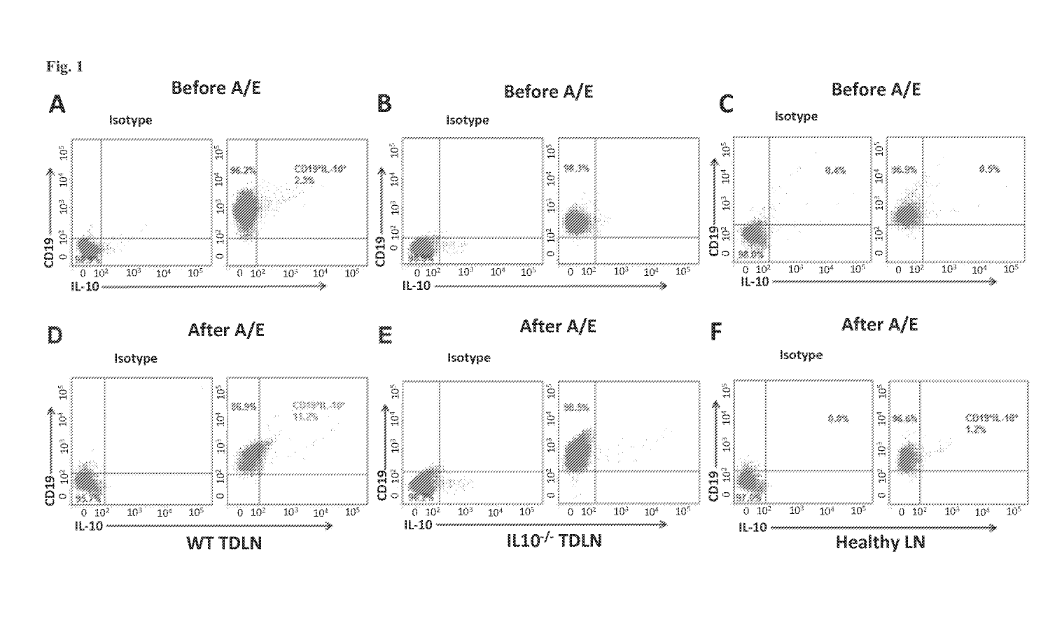

Breg cells have been found to be immunosuppressive (Mizoguchi, A., et al., Immunity 2002. 16: 219-230; Fillatreau, S., et al., Nat. Immunol. 2002. 3: 944-950; Mauri, C., et al., J. Exp. Med. 2003. 197: 489-501; Inoue, S., et al., Cancer Res. 2006. 66: 7741-7747; Schioppa, et al., Proc. Natl. Acad. Sci. USA 2011. 108: 10662-10667; Tanaba, K., et al., J. Immunol. 2009. 182: 7459-7472; DiLillo, D. J., et al., Ann. NY Acad. Sci. 2010. 1183: 38-57; Evans, J. et al., J. Immunol. 2007. 178: 7868-7878; Yanaba, K., et al., Immunity 2008. 28: 639-650; Matsushita, T., et al., J. Clin. Invest. 2008. 118: 3420-3430; Koni, P. A., et al., J. Immunol. 2013. 190: 3189-3196). To detect IL-10-producing cells in 4T1 TDLN B cells, CD19⁺ B cells were purified from WT and IL-10^{-/-} 4T1 TDLN cells, respectively. WT 4T1 TDLNs were induced as previously described (Li, Q., et al., Clin. Cancer Res. 2011. 17: 4987-4995), and the IL-10^{-/-} 4T1 TDLNs were induced by s.c. injection of 4T1 cells into the IL-10^{-/-} BALB/c mice. The CD19+ and CD19⁺IL-10⁺ Bcell populations were assessed by flow cytometry. Among these freshly purified B cells, 2-3% of the WT B cells were CD19⁺IL-10⁺ (FIG. 1A), but these cells were not detectable in the IL-10^{-/-} B cells as expected (FIG. 1B). After in vitro activation and expansion (A/E) with LPS plus anti-CD40, CD19⁺IL-10⁺ cells in WT TDLN B cells increased to 11% (FIG. 1D), while CD19⁺IL-10⁺ cells in the IL-10/B cells remained undetectable (FIG. 1E). There were almost no IL-10⁻ producing B cells in healthy LN (<1% before A/E, FIG. 1C; <2% after A/E, FIG. 1F).

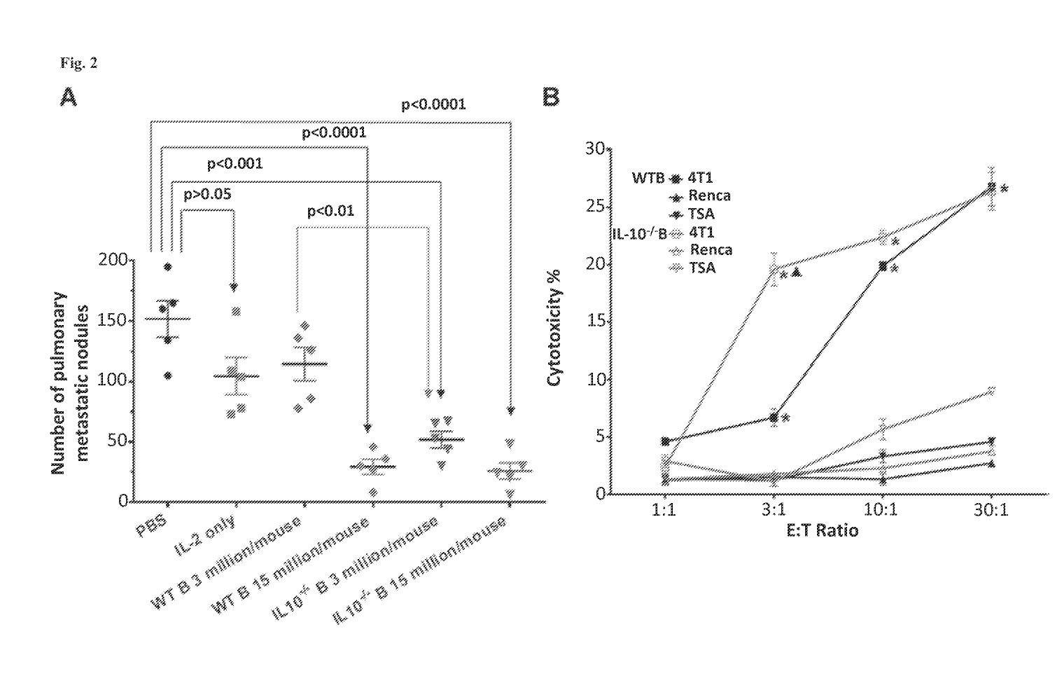

To investigate the role of IL-10-producing B cells in adoptive immunotherapy of cancer, the therapeutic efficacy of IL-10^{-/-} was compared to WT TDLN B cells. Two weeks after 4T1 tumor cell injection into the mammary fat pad, tumor-bearing WT BALB/c mice were treated with activated WT or IL-10^{-/-} 4T1 TDLN B cells. Two weeks later, mice lungs were collected to enumerate pulmonary metastases. As shown in FIG. 2A, IL-2 alone or WT 4T1 TDLN B cells at a sub-optimal low dose (3 million/mouse) had a modest, but not significant reduction in pulmonary metastases compared with PBS-treated controls. However, adoptively transferred WT 4T1 TDLN B cells at a higher dose (15 million/mouse) significantly inhibited the metastasis of 4T1 tumor cells from the injection site (mammary fat pad) to the lung, which was consistent with previous findings (Li, Q., et al., Clin. Cancer Res. 2011. 17: 4987-4995). In comparison, the higher dose (15 million/mouse) of IL-10^{-/-} 4T1 TDLN B cells demonstrated a similar antitumor activity as the higher dose of WT 4T1 TDLN B cells (p=0.7). IL-10^{-/-} B cells at the low dose (3 million/mouse) inhibited metastases significantly more effectively than WT B cells at the same low dose (p<0.01). These results indicated that IL-10^{-/-} 4T1 TDLN B cells were more potent than WT 4T1 TDLN B cells on a per cell basis in adoptive immunotherapy.

To determine the efficacy of activated IL-10^{-/-} B cells to mediate 4T1 tumor cell lysis, IL-10 and WT TDLN B cells were prepared as in FIG. 2A, and were incubated in vitro with 4T1 tumor cells and cytotoxicity was analyzed using the lactate dehydrogenase (LDH) release assay. Two other BALB/c tumors, Renca and TSA, were used for specificity controls. While neither IL-10^{-/-} nor WT 4T1 TDLN B cells killed Renca and TSA, WT 4T1 TDLN B cells killed 4T1 tumor cells in a dose-dependent manner (FIG. 2B). While at the higher E:T ratios (10:1 and 30:1), there was no significant difference in 4T1 cell killing between the IL-10^{-/-} and WT 4T1 TDLN B cells, IL-10^{-/-} 4T1 TDLN B cells mediated 4T1 cell lysis much more effectively (p<0.05) than WT 4T1 TDLN B cells at a low E:T ratio (3:1). These data indicate that TDLN B cells killed the tumor cells directly in a tumor antigen-specific manner, and that the IL-10 TDLN B cells are more potent than WT TDLN B cells in such direct killing in vitro. These data are supportive of the observation in vivo (FIG. 2A) that IL-10^{-/-} B cells are more potent antitumor effector cells than WT B cells.

IL-10 Neutralization Verifies that Adoptive Immunotherapy Using Effector TDLN B Cells is More Effective in the Absence of IL-10

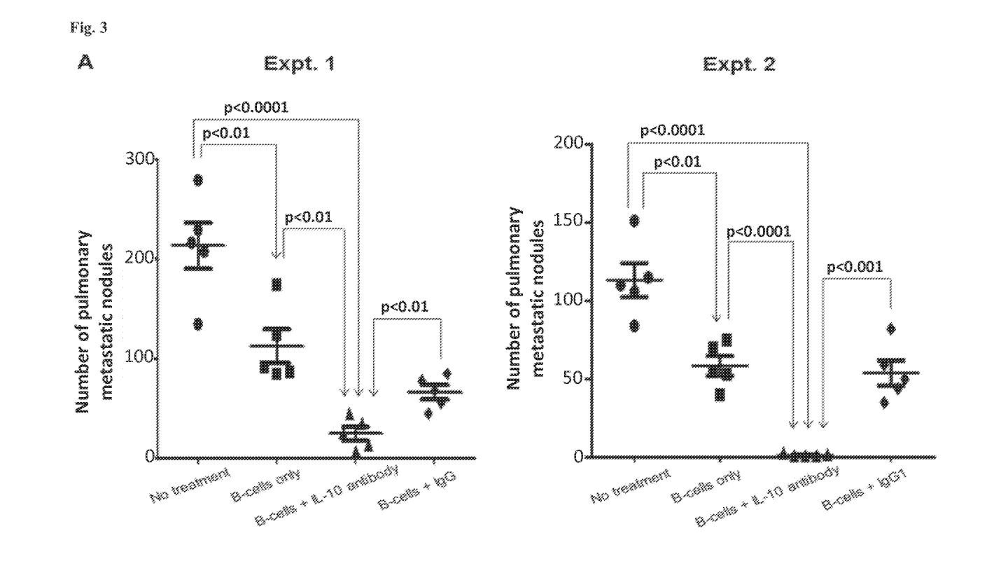

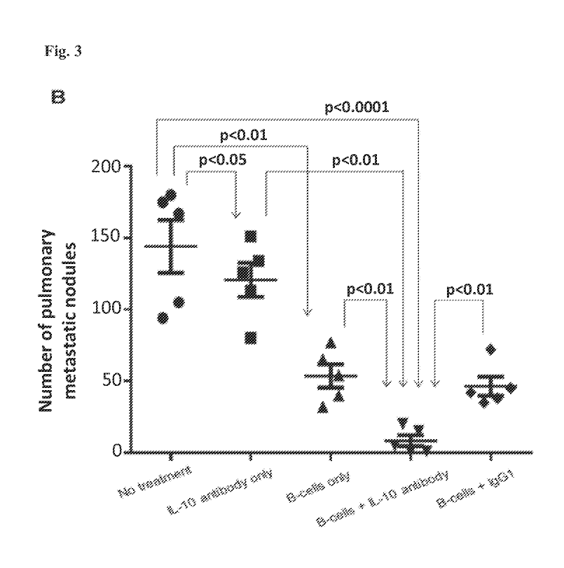

An IL-10 antibody was used to neutralize IL-10 during the adoptive immunotherapy of cancer using effector TDLN B cells. As in FIG. 2A, healthy BALB/c mice were inoculated with 4T1 cells in the mammary fat pad to induce spontaneous pulmonary metastasis and were treated 14 days later by adoptive transfer of activated WT 4T1 TDLN B cells i.v. accompanied with IL-10 or isotype control antibody administration. As revealed in FIG. 3A, with the injection of IL-10 antibody, infusion of 10×10⁶ activated B cells resulted in significantly (p<0.01) reduced spontaneous 4T1 metastases compared with the results using same number of B cells without anti-IL-10 administration (B cells only) or with IgG (Expt. 1) or IgG1 (Expt. 2) administration. In additional experiments, it was found that IL-10 antibody injection alone did not demonstrate significant antitumor effect compared with no treatment controls (FIG. 3B). Together, these studies verify that adoptive immunotherapy using effector TDLN B cells is more effective in the absence of IL-10.

### Depletion of IL-10 in B Cell Adoptive Transfer Significantly Enhances Systemic Antitumor Immunity

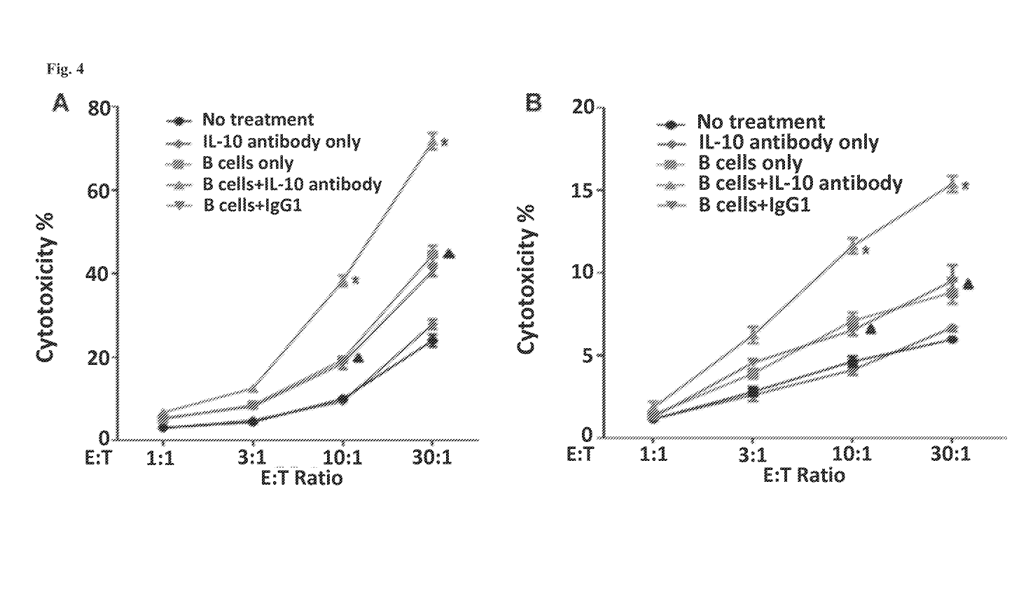

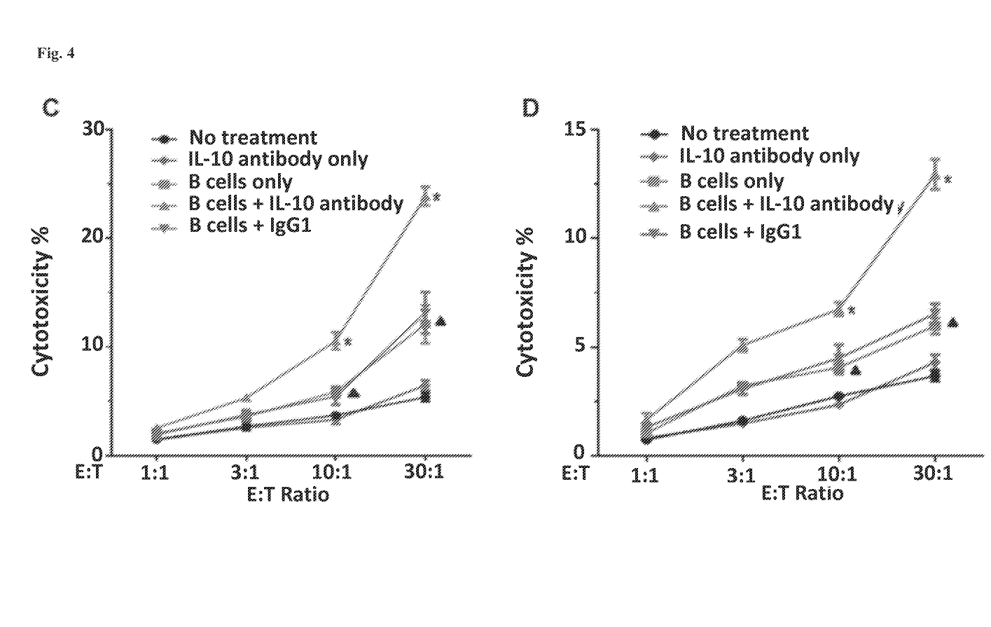

PBMCs were purified from the 4T1 tumor-bearing host subjected to WT TDLN B-cell adoptive immunotherapy with or without systemic IL-10 neutralization. T cells and B cells were purified from these PBMCs and were cultured as previously described (Li, Q., et al., J. Immunol. 2009. 183: 3195-3203; Li, Q., et al., Clin. Cancer Res. 2011. 17: 4987-4995). These cells were then analyzed for their lysis of the 4T1 cells. T cells (FIG. 4A) and B cells (FIG. 4B) generated from the PBMCs harvested from the hosts subjected to 4T1 TDLN B-cell+IL-10 antibody treatment killed 4T1 tumor cells significantly (p<0.05) more efficiently than all the controls. Splenic T- and B cells were purified from all of the five experimental groups. Both splenic T- (FIG. 4C) and splenic B cells (FIG. 4D) harvested from the hosts subjected to 4T1 TDLN B-cell+IL-10 antibody treatment lysed 4T1 tumor cells significantly (p<0.05) more efficiently than T- and B cells prepared from the hosts subjected to 4T1 TDLN B-cell alone, 4T1 TDLN B-cell+IgG1, IL-10 antibody only, or no treatment. Together, these data indicate that depletion of IL-10 in B-cell adoptive transfer significantly enhanced host systemic antitumor immunity.

### Direct Killing of 4T1 Tumor Cells by 4T1 TDLN B Cells Involves the Fas/FasL and CXCR4/CXCL12 Pathways

In the observed direct killing of 4T1 tumor cells by B cells, either by effector 4T1 TDLN B cells used for adoptive transfer (FIG. 2B), or by PBMCs and splenic B cells harvested from the host subjected to B-cell+IL-10 antibody treatment (FIGS. 4B and D), there was no complement or other effector cells (e.g. NK cells, macrophages, or neutrophils) added in the assay. Therefore, such direct killing was distinct from the traditional complement-dependent cytotoxicity or antibody-dependent cell cytotoxicity.

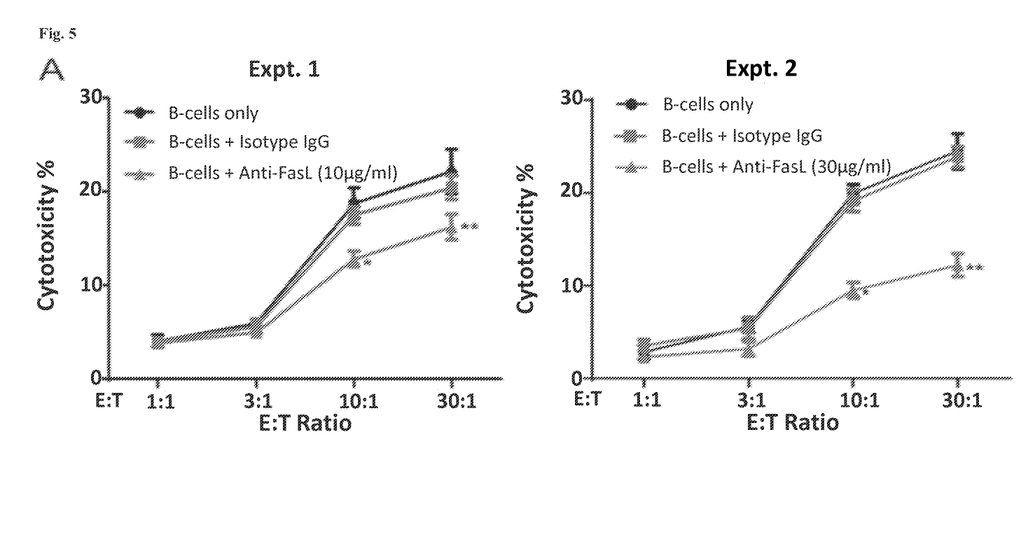

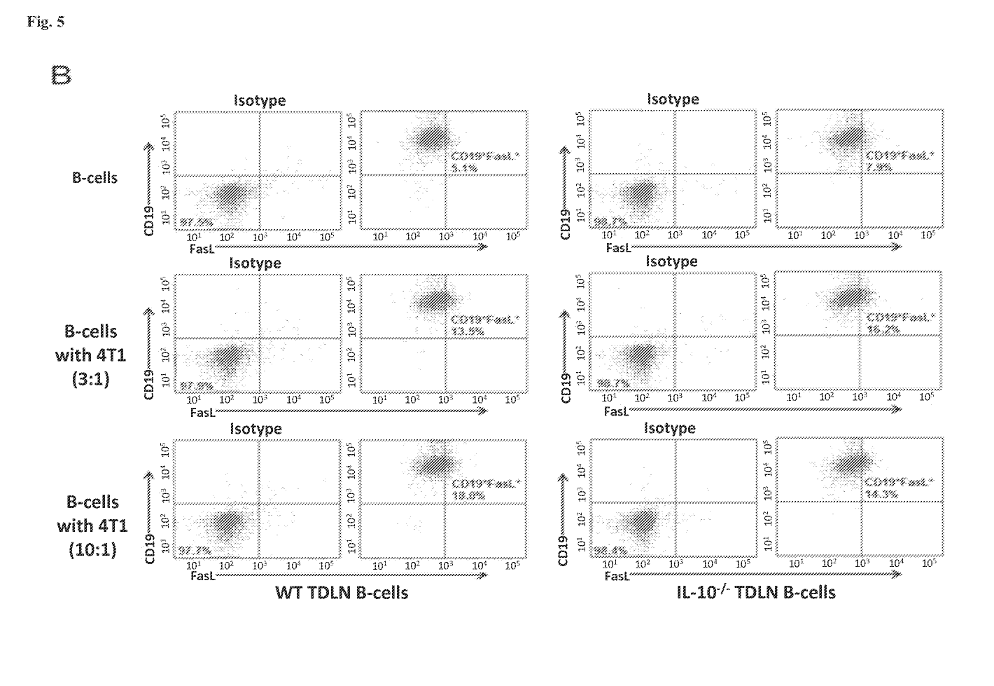

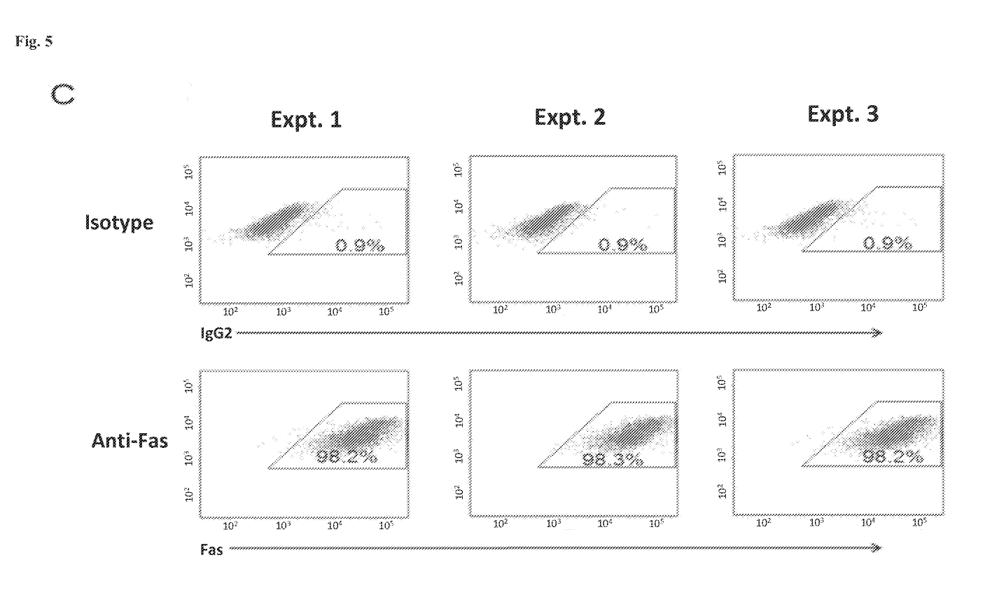

B cells have been shown to express FasL that can bind to the target cells expressing Fas resulting in target cell death (Lundy, S. K., Inflamm. Res. 2009. 58: 345-357; Hahne, M., et al., Eur. J. Immunol. 1996. 26: 721-724; Strater, J., et al., Am. J. Pathol. 1999. 154: 193-201). To examine whether 4T1 TDLN B-cell-mediated direct killing of 4T1 cells involved the Fas/FasL pathway, anti-FasL antibody was used to block FasL during the LDH release assay. When added in culture, anti-FasL antibody significantly and dose-dependently decreased the killing efficacy of TDLN B cells on 4T1 tumor cells at the E:T ratios of 10:1 and 30:1 (FIG. 5A, Expt. 1, anti-FasL=10 mg/mL; Expt. 2, anti-FasL=30 mg/mL).

FasL expression in TDLN B cells isolated from IL-10^{-/-} knockout mice was compared with WT TDLN B cells. As shown in FIG. 5B, approximately 5% of the purified and anti-CD40/LPS A/E WT TDLN B cells expressed FasL. These are the effector cells used for adoptive transfer, in vitro killing assays, and anti-FasL blockade throughout the study. As also observed in FIG. 5B, there is a similar percentage (˜8%) of the IL-10 TDLN B cells expressing FasL, showing no significant difference between these two types of B cells. It was found that when TDLN B cells were cocultured overnight with 4T1 tumor cells, the FasL expression was increased on the B cells. At WT TDLN B-cell:4T1 ratios of 3:1 and 10:1 the FasL expression on the B cells increased from 5.1 to 13.5% and 18.0%, respectively. A similar increase of FasL expression was observed on the IL-10^{-/-} TDLN B cells after their coculturing with 4T1 tumor cells in FIG. 5B. Fas expression in target 4T1 tumor cells was very high (FIG. 5C), correlating with their sensitivity to be targeted by FasL⁺ activated TDLN B cells.

### Adoptively Transferred B Cells Traffic to the Tumor and Lung In Vivo

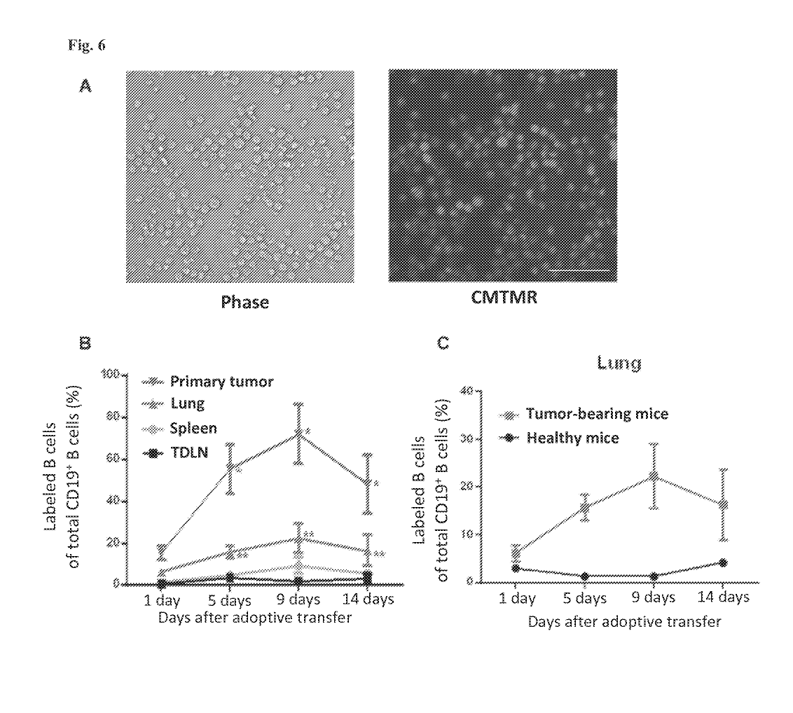

To address homing of the transferred B cells, the purified and activated/expanded TDLN B cells were pre-labeled with 10 μM Cell Tracker™ Orange CMTMR (FIG. 6A). After adoptive transfer, spleens, TDLNs, lungs, and primary tumors were harvested at specified time points to detect the labeled live B cells in these tissues. As shown in FIG. 6B, adoptively transferred B cells represented a high percentage of the total CD19⁺ B cells in the tumor site and lung peaking at 9 days posttransfer. Transferred B cells remained high in the tumor and lung on day 14 when lung metastases were examined. The results showed that transferred TDLN B cells represented a smaller fraction of the B cells found in the spleen and TDLNs but that the total numbers of transferred B cells in the spleen and TDLN were higher in comparison to those in the tumor and lung (Table 1). These data indicate that transferred B cells did not preferentially home to the tumor sites, but that they were more prone to entering sites of tumor than were endogenous B cells. This indicates that the ex vivo activation of TDLN B cells enhanced their trafficking to tumor sites, which may have enhanced their ability to prevent lung metastasis.

In the above experiments, tumor-free mice were also used as recipients of labeled TDLN B cells and very few transferred B cells were found in the lung (FIG. 6C) or in the spleen and LN of the healthy mice. These results indicate that localization and/or survival of the adoptively transferred TDLN B cells is dependent on interaction with the 4T1 tumor cells in vivo.

TABLE 1

| | Primary | | | |
|---|---|---|---|---|
| | tumor | Lung | Spleen | TDLN |
| | (×10⁴) | (×10⁴) | (×10⁴) | (×10⁴) |
| 1 day | 3.67 ± 0.38 | 1.97 ± 0.34 | 7.44 ± 0.97 | 5.33 ± 1.04^{a)} |
| 5 days | 14.23 ± 1.39 | 5.87 ± 0.77 | 24.15 ± 5.24 | 35.90 ± 2.33^{b)} |
| 9 days | 17.34 ± 0.58 | 13.41 ± 0.95 | 43.34 ± 4.34 | 28.37 ± 3.03^{c)} |
| 14 days | 16.18 ± 2.16 | 14.71 ± 1.91 | 42.06 ± 9.55 | 26.87 ± 5.56 |

^{a)}p < 0.05₃ TDLN versus lung.
^{b)}p < 0.05₃ TDLN versus primary tumor or versus lung.
^{c)}p < 0.05₃ TDLN versus primary tumor or versus lung or versus spleen

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of treating cancer, comprising:
a) isolating B cells from a subject diagnosed with cancer;
b) engineering said B cells ex vivo to express FasL and/or CXCR4; and
c) administering said engineered B cells to said subject.

2. The method of claim 1, wherein said engineering comprises genetic modification.

3. The method of claim 2, wherein said genetic modification comprises introducing a nucleic acid expressing FasL and/or CXCR4 to said cell.

4. The method of claim 3, wherein said nucleic acid is in a vector or is administered as a naked nucleic acid.

5. The method of claim 1, further comprising the step of activating said engineered B cells prior to said administering.

6. The method of claim 1, wherein said B cells are CD19⁺ B cells.

7. The method of claim 1, wherein said B cells lack IL-10 expression.

8. The method of claim 7, wherein said B cells are engineered to lack IL-10 expression.

9. The method of claim 8, wherein said engineering comprising genetic therapy to knock-out a gene expressing IL-10, or nucleic acid therapy with an siRNA, an antisense RNA, a miRNA, or a shRNA.

10. The method of claim 1, wherein said B cells are isolated from tumor draining lymph nodes, blood, or splenocytes.

11. The method of claim 1, wherein 1 million to 100 million engineered B cells are administered to said subject.

12. The method of claim 1, wherein 1 million to 5 million engineered B cells are administered to said subject.

13. The method of claim 5, wherein said B cells are activated with lipopolysaccharide (LPS) and anti-CD40 monoclonal antibody treatment.

14. The method of any one of claims 1 to 14, wherein said method further comprises the step of isolating T cells from said subject, activating said T cells ex vivo to generate activated T cells, and administering said activated T cells to said subject.

15. The method of claim 14, wherein said T-cells are activated with anti-CD3 and anti-CD28 monoclonal antibodies and IL-2.

16. The method of any one of claims 1 to 15, further comprising the administration of one or more additional cancer therapies.

17. The method of claim 16, wherein said one or more additional cancer therapies are selected from chemotherapy, radiation therapy, surgery, and immunological therapies.

18. A composition comprising a B cells comprising an exogenous FasL and/or CXCR4 gene.

19. The composition of claim 18, wherein said composition further comprises activated T cells.

20. The composition of claim 18, wherein said B cells lack a functional IL-10 gene.

21. The composition of any one of claims 18 to 20, wherein said composition is a pharmaceutical composition.

22. The use of the composition of any one of claims 18 to 21 in the treatment of cancer.

23. The use of the composition of any one of claims 18 to 21 in the preparation of a medicament for use in the treatment of cancer.
